(19) United States
(12) Patent Application Publication
(10) Pub. No.: US 2009/0143671 A1
(43) Pub. Date: Jun. 4, 2009

(54) POSITION IDENTIFYING SYSTEM, POSITION IDENTIFYING METHOD, AND COMPUTER READABLE MEDIUM

(75) Inventor: Hideyasu Ishibashi, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/327,360

(22) Filed: Dec. 3, 2008

(30) Foreign Application Priority Data
Dec. 3, 2007 (JP) 2007-312399
Dec. 4, 2007 (JP) 2007-313838
Dec. 4, 2007 (JP) 2007-313839

(51) Int. Cl. A61B 5/05

(52) U.S. Cl. 600/424

(57) ABSTRACT

Provided is a position identifying system with a simple configuration that can identify a position of an object inside a body. The position identifying system identifies a position of an object existing inside a body. The position identifying system includes a vibrating section that vibrates each of a plurality of different positions inside the body at a different timing; an image capturing section that captures a frame image of the object at each of the different timings; and a position identifying section that identifies the position of the object based on a blur amount of an image of the object in each frame image captured by the image capturing section.

### CROSS REFERENCE TO RELATED APPLICATION

The present application claims priority from Japanese Patent Applications No. 2007-312399 filed on Dec. 3, 2007, No. 2007-313838 filed on Dec. 4, 2007, and No. 2007-313839 filed on Dec. 4, 2007, the contents of which are incorporated herein by reference.

### BACKGROUND

1. Technical Field

The present invention relates to a position identifying system, a position identifying method, and a computer readable medium. In particular, the present invention relates to a position identifying system, a position identifying method, and a computer readable medium used by the position identifying system for identifying a position of an object existing inside a body.

2. Related Art

A measurement apparatus for collecting information from a living organism is known that measures detailed information concerning the organism's metabolism by propagating the wavelength of light inside the organism, as in, for example, Japanese Patent Application Publication No. 2006-218013. An optical measurement apparatus is known that obtains an absorption coefficient distribution in a direction of depth in the subject by measuring the amount of light absorbed at different distances between where the light enters and exits, as in, for example, Japanese Patent Application Publication No. 8-322821.

These two apparatuses, however, use different points for irradiation and detection, making it difficult to form an observation system.

### SUMMARY

Therefore, it is an object of an aspect of the innovations herein to provide a position identifying system, a position identifying method, and a computer readable medium, which are capable of overcoming the above drawbacks accompanying the related art. The above and other objects can be achieved by combinations described in the independent claims. The dependent claims define further advantageous and exemplary combinations of the innovations herein.

According to a first aspect related to the innovations herein, one exemplary position identifying system may include a position identifying system that identifies a position of an object existing inside a body, comprising a vibrating section that vibrates each of a plurality of different positions inside the body at a different timing; an image capturing section that captures a frame image of the object at each of the different timings; and a position identifying section that identifies the position of the object based on a blur amount of an image of the object in each frame image captured by the image capturing section.

According to a second aspect related to the innovations herein, one exemplary position identifying method may include a position identifying method for identifying a position of an object existing inside a body, comprising vibrating each of a plurality of different positions inside the body at a different timing; capturing a frame image of the object at each of the different timings; and identifying the position of the object based on a blur amount of an image of the object in each frame image captured during the image capturing.

According to a third aspect related to the innovations herein, one exemplary computer readable medium may include a computer readable medium storing thereon a program causing a position identifying system that identifies a position of an object existing inside a body to function as a vibrating section that vibrates each of a plurality of different positions inside the body at a different timing; an image capturing section that captures a frame image of the object at each of the different timings; and a position identifying section that identifies the position of the object based on a blur amount of an image of the object in each frame image captured by the image capturing section.

According to a fourth aspect related to the innovations herein, one exemplary position identifying system may include a position identifying system that identifies a position of an object existing inside a body, comprising a vibrating section that vibrates the body; an image capturing section that captures a frame image of the object after the body is vibrated; and a position identifying section that identifies the position of the object inside the body based on a blur amount of an image of the object in the frame image captured by the image capturing section.

According to a fifth aspect related to the innovations herein, one exemplary position identifying method may include a position identifying method for identifying a position of an object existing inside a body, comprising vibrating the body; capturing a frame image of the object after the body is vibrated; and identifying the position of the object inside the body based on a blur amount of an image of the object in the frame image captured by the image capturing section.

According to a sixth aspect related to the innovations herein, one exemplary computer readable medium may include a computer readable medium storing thereon a program causing a position identifying system that identifies a position of an object existing inside a body to function as a vibrating section that vibrates the body; an image capturing section that captures a frame image of the object after the body is vibrated; and a position identifying section that identifies the position of the object inside the body based on a blur amount of an image of the object in the frame image captured by the image capturing section.

According to a seventh aspect related to the innovations herein, one exemplary position identifying system may include a position identifying system that identifies a position of an object existing inside a body, comprising a vibrating section that vibrates the body; an image capturing section that captures a frame image of the object when the body is vibrated and also when the body is not vibrated; and a position identifying section that identifies the position of the object inside the body based on a blur amount of an image of the object in the frame image captured by the image capturing section.

According to an eighth aspect related to the innovations herein, one exemplary position identifying method may include a method for identifying a position of an object existing inside a body, comprising vibrating the body; capturing a frame image of the object when the body is vibrated and also when the body is not vibrated; and identifying the position of the object inside the body based on a blur amount of the image of the object in each frame image captured during the image capturing.

According to a ninth aspect related to the innovations herein, one exemplary computer readable medium may include a computer readable medium storing thereon a program causing a position identifying system that identifies a position of an object existing inside a body to function as a vibrating section that vibrates the body; an image capturing section that captures a frame image of the object when the body is vibrated and also when the body is not vibrated; and a position identifying section that identifies the position of the object inside the body based on a blur amount of the image of the object in each frame image captured by the image capturing section.

The summary clause does not necessarily describe all necessary features of the embodiments of the present invention. The present invention may also be a sub-combination of the features described above. The above and other features and advantages of the present invention will become more apparent from the following description of the embodiments taken in conjunction with the accompanying drawings.

### BRIEF DESCRIPTION OF THE DRAWINGS

FIG. 1 shows an exemplary configuration of a position identifying system 10 according to the present embodiment, along with a subject 20.

FIG. 2 shows an exemplary configuration of the image processing section 140.

FIG. 3 shows an exemplary configuration of the vibrating section 133.

FIG. 4 shows a method performed by the position identifying section 230 for detecting depth.

FIG. 5 is a distance calculation table stored in the distance calculating section 236.

FIG. 6 shows an exemplary frame image 600 corrected by the image correcting section 220.

FIG. 7 shows an exemplary method of depth detection performed by the position identifying section 230.

FIG. 8 shows another exemplary method of depth detection performed by the position identifying section 230.

FIG. 9 shows exemplary frame images 901 and 902 captured when the vibrating section 133 vibrates different positions.

FIG. 10 shows an exemplary hardware configuration of the position identifying system 10 according to the present embodiment.

### DESCRIPTION OF EXEMPLARY EMBODIMENTS

Hereinafter, some embodiments of the present invention will be described. The embodiments do not limit the invention according to the claims, and all the combinations of the features described in the embodiments are not necessarily essential to means provided by aspects of the invention.

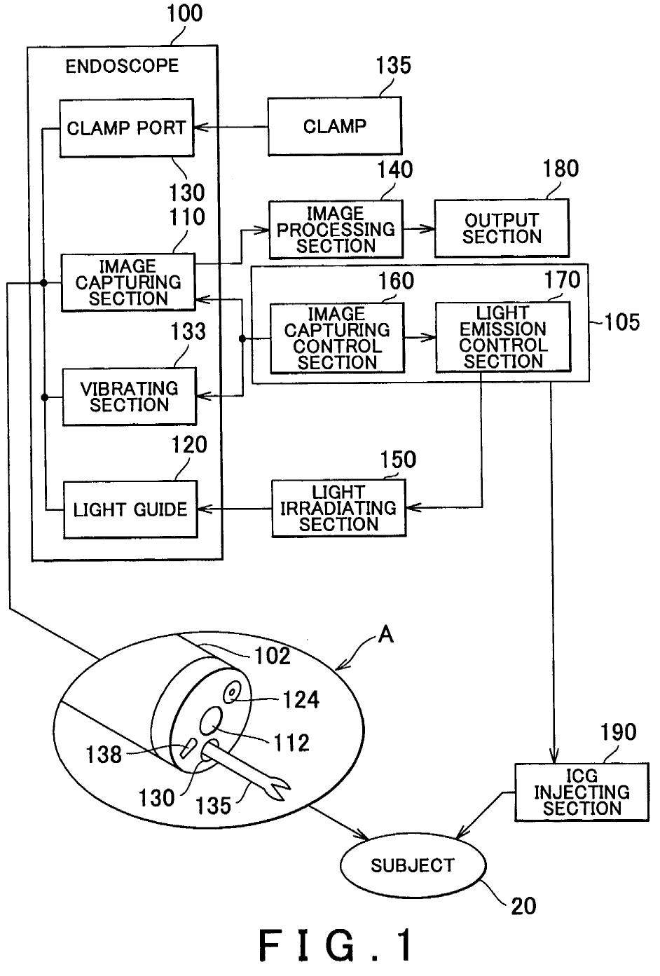

FIG. 1 shows an exemplary configuration of a position identifying system 10 according to the present embodiment, along with a subject 20. The position identifying system 10 identifies a position of an object existing inside a body. The position identifying system 10 is provided with an endoscope 100, an image processing section 140, an output section 180, a control section 105, a light irradiating section 150, and an ICG injecting section 190. In FIG. 1, the section “A” is an enlarged view of the tip 102 of the endoscope 100. The control section 105 includes an image capturing control section 160 and a light emission control section 170.

The ICG injecting section 190 injects indocyanine green (ICG), which is a luminescent substance, into the subject 20, which is an example of the body in the present invention. The ICG is an example of the luminescent substance in the present embodiment, but the luminescent substance may instead be a different fluorescent substance. The ICG is excited by infra-red rays with a wavelength of 750 nm, for example, to emit broad spectrum fluorescence centered at 810 nm.

If the subject 20 is a living organism, the ICG injecting section 190 injects the ICG into the blood vessels of the organism through intravenous injection. The position identifying system 10 captures images of the blood vessels in the organism from the luminescent light of the ICG. This luminescent light includes fluorescent light and phosphorescent light. The luminescent light, which is an example of the light from the body, includes chemical luminescence, frictional luminescence, and thermal luminescence, in addition to the luminescence from the excitation light or the like. The blood vessels are examples of the objects in the present invention.

The ICG injecting section 190 is controlled by the control section 105, for example, to inject the subject 20 with ICG such that the ICG density in the organism is held substantially constant. The subject 20 may be a living organism such as a person. Objects such as blood vessels exist inside the subject 20. The position identifying system 10 of the present embodiment detects the position, i.e. depth, of objects existing below the surface of the subject 20, where the surface may be the inner surface of an organ. The position identifying system 10 corrects the focus of the frame image of the object according to the detected position. The body in this invention may be an internal organ of a living organism, such as the stomach or intestines, or may be an inorganic including natural bodies such as ruins and inorganic bodies such as industrial products.

The endoscope 100 includes an image capturing section 110, a light guide 120, a vibrating section 133, and a clamp port 130. The tip 102 of the endoscope 100 includes an objective lens 112, which is a portion of the image capturing section 110, an irradiation aperture 124, which is a portion of the light guide 120, and a nozzle 138, which is a portion of the vibrating section 133.

A clamp 135 is inserted into the clamp port 130, and the clamp port 130 guides the clamp 135 to the tip 102. The tip of the clamp 135 may be any shape. Instead of the clamp, various types of instruments for treating the organism can be inserted into the clamp port 130. The nozzle 138 ejects water or air.

The light irradiating section 150 generates the light to be radiated from the tip 102 of the endoscope 100. The light generated by the light irradiating section 150 includes irradiation light that irradiates the subject 20 and excitation light, such as infra-red light, that excites the luminescent substance inside the subject 20 such that the luminescent substance emits luminescent light. The irradiation light may include a red component, a green component, and a blue component.

The image capturing section 110 captures a frame image based on the reflected light, which is the irradiation light reflected by the object, and the luminescent light emitted by the luminescent substance. The image capturing section 110 may include an optical system and a two-dimensional image capturing device such as a CCD, or may include the lens 112 in an optical system. If the luminescent substance emits infra-red light, the image capturing section 110 can capture an infra-red light frame image. If the light irradiating the object contains red, green, and blue components, i.e. if the irradiation light is white light, the image capturing section 110 can capture a visible light frame image.

The light from the object may be luminescent light such as fluorescent light or phosphorescent light emitted by the luminescent substance in the object, or may be the irradiation light that reflects from the object or that passes through the object. In other words, the image capturing section 110 captures a frame image of the object using the light emitted by the luminescent substance inside of the object, the light reflected by the object, or the light passing through the object.

The image capturing section 110 can capture a frame image of the object using various techniques that do not involve receiving light from the object. For example, the image capturing section 110 can capture a frame image of the object using electromagnetic radiation such as X-rays or γ-rays, radiation including particle beams such as alpha rays, or the like. The image capturing section 110 may capture the frame image of the object using sound waves, electrical waves, or electromagnetic waves having various wavelengths.

The light guide 120 may be formed of optical fiber. The light guide 120 guides the light emitted by the light irradiating section 150 to the tip 102 of the endoscope 100. The light guide 120 can have the irradiation aperture 124 provided in the tip 102. The light emitted by the light irradiating section 150 passes though the irradiation aperture 124 to irradiate the subject 20.

The image processing section 140 processes the image data acquired from the image capturing section 110. The output section 180 outputs the image data processed by the image processing section 140. The image capturing control section 160 controls the image capturing by the image capturing section 110. The light emission control section 170 is controlled by the image capturing control section 160 to control the light irradiating section 150. For example, when the image capturing section 110 performs image capturing alternately with infra-red light and irradiation light, the light emission control section 170 controls the image capturing section 110 to synchronize the timing of the image capturing with the emission timing of the infra-red light and the irradiation light.

The vibrating section 133 causes the body to vibrate. For example, the vibrating section 133 causes the surface of the subject 20 to vibrate by discharging air from the tip of the nozzle 138. As another example, the vibrating section 133 can cause the surface of the subject 20 to vibrate using sound waves or supersonic waves. During vibration, the image processing section 140 identifies the depth of the blood vessels from the surface of the subject 20 based on the amount of blur in portions of the frame image captured by the image capturing section 110. The vibrating section 133 desirably causes the surface of the body to vibrate in a manner to include movement in a direction perpendicular to the frame image capturing direction of the image capturing section 110.

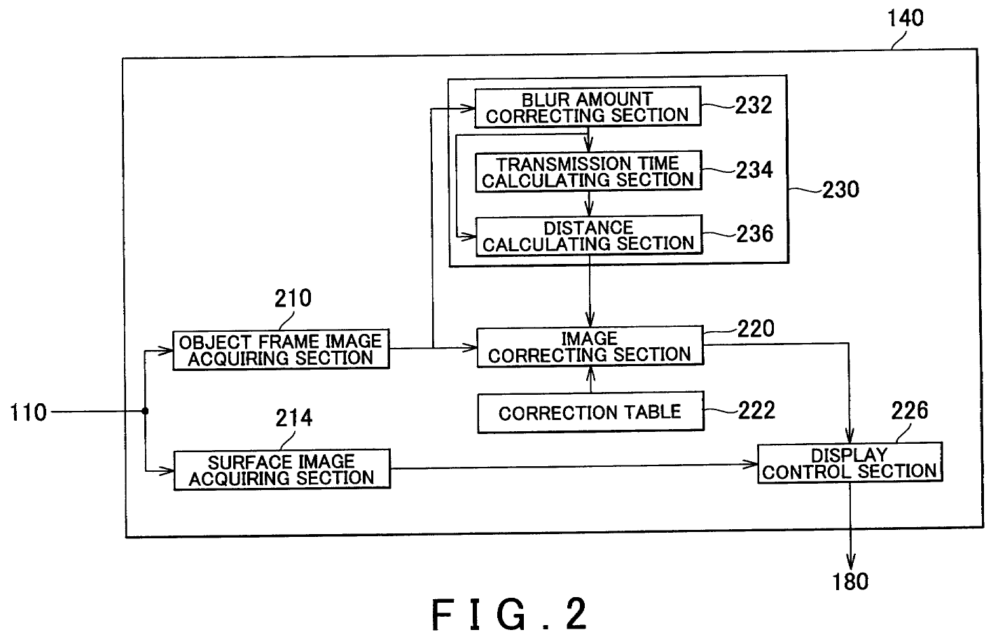

FIG. 2 shows an exemplary configuration of the image processing section 140. The image processing section 140 includes an object frame image acquiring section 210, a surface image acquiring section 214, an image correcting section 220, a correction table 222, a display control section 226, and a position identifying section 230. The position identifying section 230 includes a blur amount calculating section 232, a transmission time calculating section 234, and a distance calculating section 236.

The object frame image acquiring section 210 acquires an object frame image, which is a frame image based on the light from the object, i.e. the blood vessel, inside the subject 20. More specifically, the frame image captured by the image capturing section 110 based on the light from the object is acquired as the object frame image. The image capturing section 110 captures the frame image of the object after the body is caused to vibrate. The object frame image acquiring section 210 acquires the object frame image captured by the image capturing section 110.

If the light from the object is luminescent light emitted by the luminescent substance, the object frame image acquired by the object frame image acquiring section 210 includes an image of an object in a range extending as deep from the surface as the excitation light exciting the luminescent substance can penetrate. For example, if the luminescent substance excitation light radiated from the tip 102 of the endoscope 100 has a wavelength of 750 nm, the excitation light can penetrate relatively deeply into the subject 20, i.e. to a depth of several centimeters. Therefore, the object frame image acquired by the object frame image acquiring section 210 can include the image of a blood vessel that is relatively deep in the subject 20. The blood vessel image is an example of the images of the object in the object frame image of the present invention.

The luminescent substance existing within the depth to which the excitation light can penetrate is excited by the excitation light, so that the object frame image acquired by the object frame image acquiring section 210 includes the image of the blood vessel existing within the depth to which the excitation light can penetrate. The image of the blood vessel becomes more blurred for a blood vessel that is deeper because the fluorescent light from the blood vessels is scattered by the subject 20.

The surface image acquiring section 214 acquires a surface image of the body. That is, the surface image acquiring section 214 acquires an image equivalent to what can be seen by the eye. For example, the surface image acquiring section 214 acquires, as the surface image, an image captured by the image capturing section 110 based on the irradiation light reflected from the surface of the body.

The position identifying section 230 identifies the position of the objects in the body based on the amount of blurring of the object image in the object frame image acquired by the object frame image acquiring section 210. More specifically, the blur amount calculating section 232 calculates the blur amount of the object image in the object frame image.

The transmission time calculating section 234 calculates a transmission time that indicates the length of the period from when the body begins to vibrate to when the vibration reaches the object, based on the blur amount of the object image in the object frame image as calculated by the blur amount calculating section 232. For example, the transmission time calculating section 234 calculates the transmission time to be the length of the period from when the body begins to vibrate to when the blur amount caused by the vibration exceeds a predetermined value.

The distance calculating section 236 calculates a distance from the position of the vibration in the body caused by the vibrating section 133 to the position of the object, based on the transmission time calculated by the transmission time calculating section 234. For example, the distance calculating section 236 can calculate longer distances for longer transmission times calculated by the transmission time calculating section 234. The distance calculating section 236 can calculate a distance from the position of the body that is vibrated by the vibrating section 133 based on the transmission time and a transmission speed that indicates the distance that the vibration travels per unit time.

When the vibrating section 133 vibrates the body from the surface, the transmission time calculating section 234 may calculate the transmission time to be the period from when the vibrating section 133 vibrates the surface to when the blur amount caused by the vibration becomes greater than a preset value. In this case, the distance calculating section 236 may calculate the depth of the object in relation to the surface based on the transmission time calculated by the transmission time calculating section 234.

The image correcting section 220 corrects the spread of the object image in the object frame image based on the depth identified by the position identifying section 230. As described above, the images of the objects are blurred due to scattering caused by the body between the object and the surface. The image correcting section 220 corrects the blur according to the depth of the object from the surface identified by the position identifying section 230.

More specifically, the correction table 222 stores correction values for correcting the spread of the object image in the object frame image, in association with the depth of the object. The image correcting section 220 corrects the spread of the object image in the object frame image based on the correction values stored in the correction table 222 and the depth of the object calculated by the position identifying section 230.

The display control section 226 controls the display of the frame image corrected by the image correcting section 220 according to the depth of the objects. For example, the display control section 226 changes the color or brightness of the object image in the object frame image corrected by the image correcting section 220, according to the depth of the object.

The position identifying section 230 may identify the depth of each of a plurality of objects from the surface. More specifically, the transmission time calculating section 234 may calculate a transmission time for each of the plurality of objects. The distance calculating section 236 may calculate the depth of each object from the surface based on the transmission time calculated by the transmission time calculating section 234. The image correcting section 220 may correct the spread of the object images in the object frame image based on the depth of each object.

The frame image corrected by the image correcting section 220 is provided to the output section 180 to be displayed. The display control section 226 controls the display of the frame image corrected by the image correcting section 220 according to the depth of each object. For example, the display control section 226 may change the color or brightness of each object in the object frame image corrected by the image correcting section 220, based on the depth of each object. The display control section 226 may instead display characters or the like indicating the depth of each object in association with the corrected frame image.

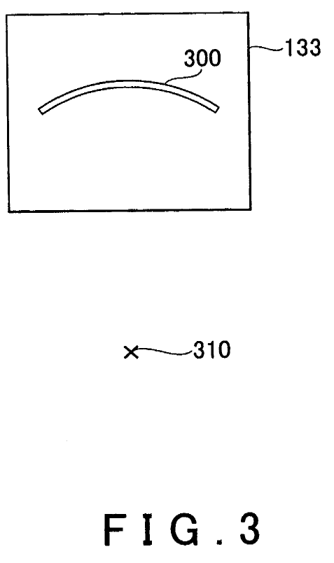

FIG. 3 shows an exemplary configuration of the vibrating section 133. The vibrating section 133 includes a vibration generating section 300. The vibration generating section 300 can generate a vibration wave centered on a focal point 310. By changing the position of the vibration generating section 300, the vibration generating section 300 can generate vibration waves at a plurality of different positions and in different directions. The vibration generating section 300 may be a supersonic wave oscillator that can generate a supersonic wave centered on the focal point 310.

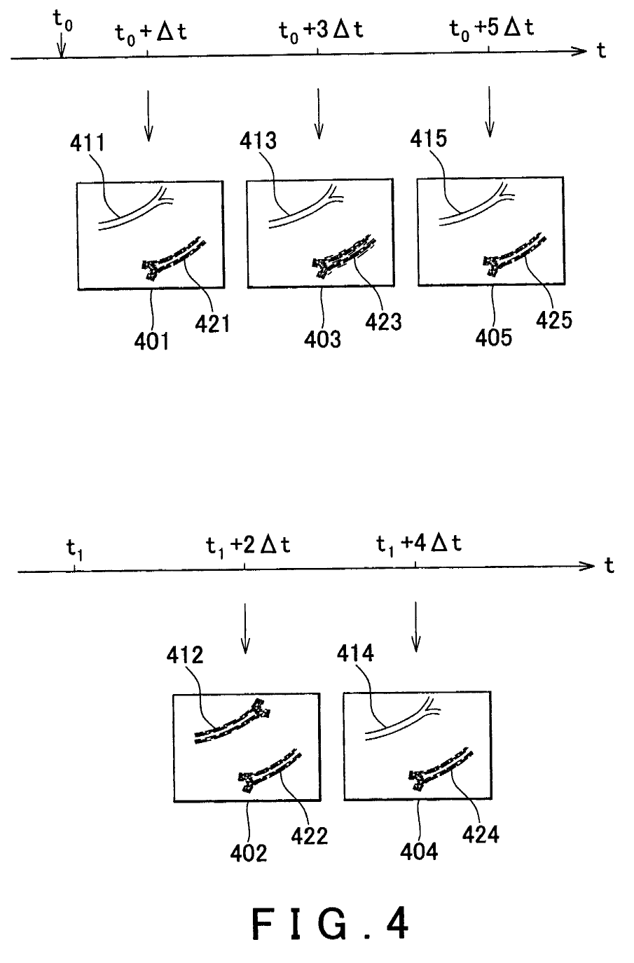

FIG. 4 shows a method performed by the position identifying section 230 for detecting depth. The vibrating section 133 vibrates the surface of the subject 20 at the time t0. At intervals of 2Δt beginning at t0+Δt, the image capturing section 110 captures frame images of the object. In FIG. 4, the image capturing section 110 captures the frame image 401, the frame image 403, and the frame image 405 at the times t0+Δt, t0+3Δt, and t0+5Δt, respectively.

At the time t1, which is not within the period during which the image capturing section 110 captures the frame images 401, 403, and 405, the surface of the subject 20 is vibrated. The image capturing section 110 captures frame images of the object at intervals of 2Δt beginning at the time t1+2Δt. In FIG. 4, the image capturing section 110 captures the frame image 402 and the frame image 404 at the times t1+2Δt and t1+4Δt, respectively.

By capturing the series of frame images described above several times, the image capturing section 110 can capture frame images of the object at intervals of Δt, beginning when the vibrating section 133 begins the vibration. The object frame image acquiring section 210 acquires the frame images 401 to 405 of the object captured by the image capturing section 110.

The frame image 401 includes the blood vessel image 411 and the blood vessel image 421, the frame image 403 includes the blood vessel image 413 and the blood vessel image 423, the frame image 405 includes the blood vessel image 415 and the blood vessel image 425, the frame image 402 includes the blood vessel image 412 and the blood vessel image 422, and the frame image 404 includes the blood vessel image 414 and the blood vessel image 424. In FIG. 4, the blood vessel shown by the blood vessel images 421 to 425 is positioned deeper than the blood vessel shown by the blood vessel images 411 to 415. Accordingly, as shown in FIG. 4, the blood vessel image 421 has a greater blur amount than the blood vessel image 411 at the time t0+Δ when the vibration has not yet reached the blood vessel shown by the blood vessel image 421.

The blur amount calculating section 232 calculates the blur amount of each of the blood vessel images 411 to 415 and 421 to 425 in the frame images 401 to 405. More specifically, the blur amount calculating section 232 calculates the blur amount in a border region between the object and another region. The blur amount may be the amount that the object image expands in the border region. The spread of the object image can be evaluated by the amount of spatial change in the brightness value of a specified color included in the object. The amount of spatial change in the brightness value may be a half-value width or a spatial derivative value of the spatial distribution.

The transmission time calculating section 234 identifies the blood vessel image 412 as having the greatest blur amount from among the blood vessel images 411 to 415 and also as having a blur amount greater than a preset value, based on the blur amounts calculated by the blur amount calculating section 232. The transmission time calculating section 234 identifies the time t+2Δ as the time at which the frame image 402 including the blood vessel image 412 is captured. The transmission time calculating section 234 then detects the transmission time from the surface to the blood vessel shown by the blood vessel images 411 to 415 to be the time difference of 2Δt between the time t1 at which the vibrating section 133 vibrated the surface of the subject 20 and the time t+2Δat which the frame image 402 is captured.

The blood vessel image 423 has the greatest amount of blur from among the blood vessel images 421 to 425. Accordingly, in the same way as described for the blood vessel images 411 to 415, the transmission time calculating section 234 calculates the transmission time from the surface to the blood vessel shown by the blood vessel images 421 to 425 to be the time difference of 3Δt, based on the amount of blur in the blood vessel images 421 to 425 detected by the blur amount calculating section 232.

The above example describes the operation of each element when the image capturing section 110 captures frame images of the object in two separate series, based on the image capture rate of the image capturing section 110, the speed at which the vibration moves through the subject 20, and the desired depth resolution. If the depth resolution, which is determined by the speed at which the vibration moves through the subject 20 and the image capture rate of the image capturing section 110, is greater than or equal to the required depth resolution, the image capturing section 110 may perform one series of image capturing.

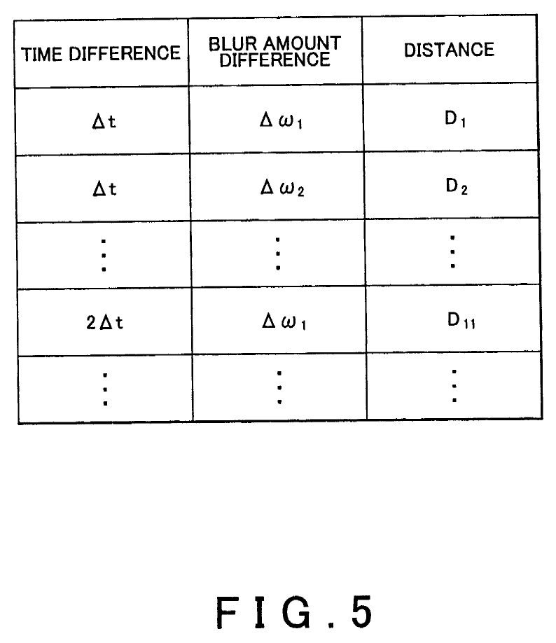

FIG. 5 is a table of information stored in the distance calculating section 236. The distance calculating section 236 stores the distance in association with the time difference and the blur amount difference in the distance calculation table of FIG. 5. As described in relation to FIG. 4, the time difference indicates the difference between (i) the time at which the vibrating section 133 begins vibrating the surface and (ii) the time at which the frame image containing the blood vessel image having the greatest blur amount is captured. The blur amount difference indicates the difference in the blur amount between the maximum blur amount of the blood vessel image and the blur amount of the blood vessel image at a time when there is no vibration or when the vibration has not yet reached the blood vessel. In the example of FIG. 5, the half-value width of the blood vessel image at a border between the blood vessel and another region indicates the blur amount, and the difference in this blur amount Δw indicates the blur amount difference.

The distance calculating section 236 calculates the distance from the surface to each blood vessel based on the transmission time calculated by the transmission time calculating section 234 and the information stored in the distance calculating table. More specifically, the distance calculating section 236 calculates the distance from the surface to each blood vessel to be the distance stored in association with the corresponding transmission time calculated by the transmission time calculating section 234.

The distance calculating section 236 may calculate the distance from the surface to each blood vessel further based on the difference between the maximum blur amount and the blur amount of the blood vessel image when there is no vibration, in addition to the transmission time. Using the blood vessel shown by the blood vessel images 411 to 415 as an example, the distance calculating section 236 may calculate the distance from the surface to the blood vessel to be the distance stored in association with the time difference Δt and the difference between the blur amount of the blood vessel image 411 and the blur amount of the blood vessel image 412. The distance calculating section 236 can increase the depth resolution by calculating the distance based on the time difference and the blur amount difference.

If the image capturing section 110 captures frame images of the objects both when the body is vibrating and when the body is not vibrating, the position identifying section 230 can identify the position of the objects inside the body based on the blur amounts of the object images in each object frame image captured by the image capturing section 110. More specifically, the position identifying section 230 identifies the position of the objects inside the body based on the difference between the blur amount of the object images when the body is vibrating and the blur amount of the object images when the body is not vibrating. The position identifying section 230 can identify the position of the objects inside the body based on this blur amount difference and the information stored in the distance calculation table described above. The position identifying section 230 can identify the position of the objects to be further away from the position on the body vibrated by the vibrating section 133 when the blur amount difference is smaller.

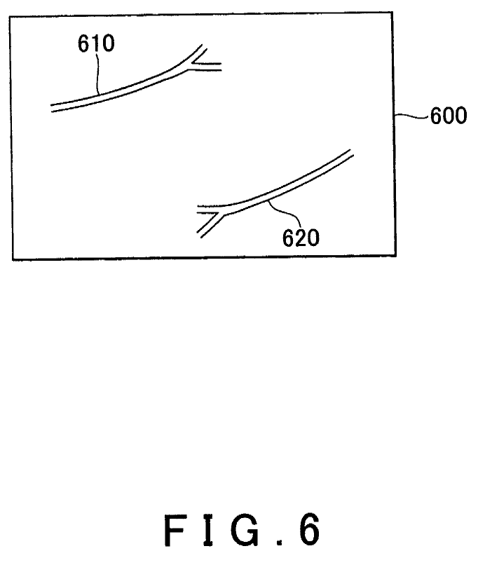

FIG. 6 shows an exemplary frame image 600 corrected by the image correcting section 220. The image correcting section 220 may correct the frame image by shrinking the spread of each blood vessel image in the frame image acquired by the object frame image acquiring section 210, according to the depth of the blood vessel detected by the position identifying section 230.

For example, the image correcting section 220 achieves the blood vessel image 620 by applying an image conversion to the blood vessel image 421 to correct the spread. More specifically, the image correcting section 220 stores a point-spread function having the depth of the blood vessel as a parameter. The point-spread function indicates the point-spread caused by the dispersion experienced by a point light source traveling to the surface. The image correcting section 220 achieves the blood vessel image 620 in which the spread of the blood vessel image is corrected by applying a filtering process to the blood vessel image 421. This filtering process uses an inverse filter of a point-spread function determined according to the depth of the blood vessel. The correction table 222 may store the inverse filter, which is an example of a correction value, in association with the depth of the object.

Since the blood vessel images in the frame image captured by the image capturing section 110 are corrected by the position identifying system 10 of the present embodiment in this way, a frame image containing clear blood vessel images 610 and 620 can be obtained. The display control section 226 causes the output section 180 to display the depth from the surface by changing the color or the shading of the blood vessel image 610 and the blood vessel image 620 in the frame image 600 according to the depth of each blood vessel. The display control section 226 may cause the output section 180 to display a combination of the frame image corrected by the image correcting section 220 and the surface image acquired by the surface image acquiring section 214. More specifically, the display control section 226 may overlap the surface image onto the frame image corrected by the image correcting section 220, and cause the output section 180 to display this combination.

The position identifying system 10 of the present embodiment enables a doctor who is watching the output section 180 while performing surgery, for example, to clearly view images of the internal blood vessels 610 and 620, and also enables the doctor to see information concerning the depth of the blood vessels.

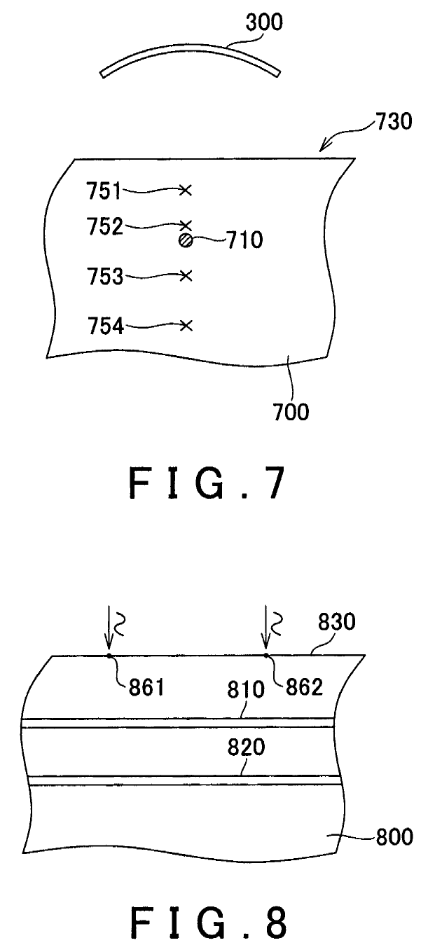

FIG. 7 shows an exemplary method of depth detection performed by the position identifying section 230. The vibrating section 133 generates a vibration wave from the vibration generating section 300 and sequentially moves the focal point of the vibration generating section 300 to positions 751, 752, 753, and 754 at different depths in the body. In this way, the vibrating section 133 generates each wave at a different timing and converging at a different position, thereby vibrating each different position in the body at a different timing.

The image capturing section 110 captures the frame image of the object at each of the different timings. The position identifying section 230 identifies the position of objects near the position of the body vibrated by the vibrating section 133 at the timing of the capture of a frame image including a frame image of an object having a blur amount greater than the preset value.

For example, the amount calculating section 232 calculates this blur amount from the blood vessel image indicating the blood vessel 710 included in each of the frame images captured by the image capturing section 110 while each of the positions 751, 752, 753, and 754, respectively, are vibrated by the vibrating section 133. The distance calculating section 236 identifies the frame image that includes the blood vessel image calculated as having the greatest blur amount by the blur amount calculating section 232. The distance calculating section 236 then determines that a blood vessel exists near the position that is vibrated by the vibrating section 133 when the identified frame image is captured.

In the example of FIG. 7, the blood vessel image showing the blood vessel 710 is expected to have a greater blur amount in the frame image captured when the position 752 is vibrated than in the frame images captured when other positions are vibrated. Therefore, the distance calculating section 236 identifies the position of the blood vessel 710 as being near the position 752. The distance calculating section 236 calculates the depth of the blood vessel from the surface 730 to be the distance from the surface 730 to the position 752.

In addition to calculating the depth of the blood vessel 710, the distance calculating section 236 may calculate the certainty of the calculated depth. For example, the distance calculating section 236 determines that the blood vessel 710 exists between (i) the midpoint between the position 751 and the position 752 and (ii) the midpoint between the position 752 and the position 753. The distance calculating section 236 sets the region between the two midpoints as having the greatest certainty near the position 752 in the distance certainty distribution. The image correcting section 220 may use the certainty distribution calculated by the distance calculating section 236 to correct the spread of the blood vessel image.

The image processing section 140 detects a plurality of blood vessels in the frame images by analyzing the frame images captured by the image capturing section 110. The position identifying section 230 identifies the position of each blood vessel in the target area of the image capturing by the image capturing section 110. The vibrating section 133 causes vibrations at different depths from the surface 730 at each identified position of a blood vessel. In this way, the position identifying section 230 can calculate the depth of each of the plurality of blood vessels.

As described above, the vibrating section 133 causes vibrations at a plurality of different positions in the body at different timings. The position identifying section 230 identifies the positions of the objects based on the blur amount of the object images in each frame image captured by the image capturing section 110.

FIG. 8 shows another exemplary method of depth detection performed by the position identifying section 230. The vibrating section 133 begins the vibration after sequentially aligning the focal point of the vibration generating section 300 with a first position 861 and a second position 862. In this way, the vibrating section 133 can vibrate the first position 861 and the second position 862 on the surface 830 of the body 800.

The image capturing section 110 captures a frame image of the objects when (i) the first position 861 is vibrated without vibrating the second position 862 and (ii) when the second position 862 is vibrated without vibrating the first position 861. The position identifying section 230 identifies the position of the objects inside the body based on the difference between (i) the blur amount of the object images when the first position 861 is vibrated without vibrating the second position 862 and (ii) the blur amount of the object images when the second position 862 is vibrated without vibrating the first position 861.

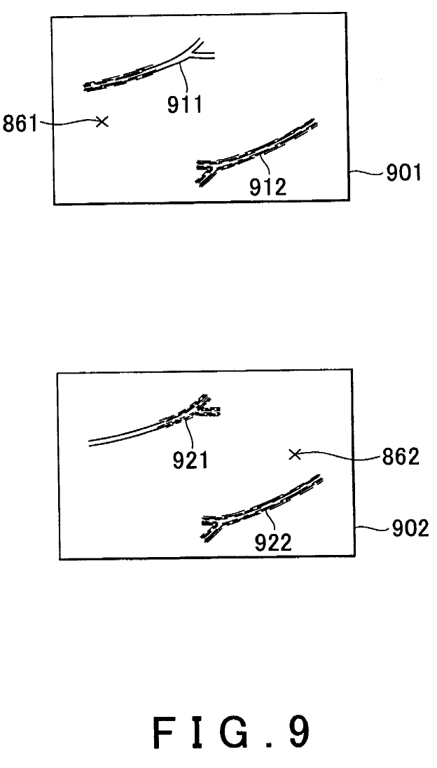

FIG. 9 shows exemplary frame images 901 and 902 captured when the vibrating section 133 vibrates different positions. The frame image 901 is captured by the image capturing section 110 when the vibrating section 133 vibrates the position 861, and the frame image 902 is captured by the image capturing section 110 when the vibrating section 133 vibrates the position 862. The blood vessel image 911 in the frame image 901 and the blood vessel image 921 in the frame image 902 show the blood vessel 810, and the blood vessel image 912 in the frame image 901 and the blood vessel image 922 in the frame image 902 show the blood vessel 820.

The blur amount of the portion of the blood vessel image 911 near the position 861 is greater than the blur amount of the portion of the blood vessel image 911 further form the position 861. On the other hand, the blur amount of the portion of the blood vessel image 921 near the position 862 is greater than the blur amount of the portion of the blood vessel image 921 further from the position 862.

The difference between the blur amounts of the portions of the blood vessel image 912 and the blood vessel image 922 near the position 861 and the position 862 is less than the difference between the blur amounts at different portions of the blood vessel image 911 and the blood vessel image 921. In this case, the distance calculating section 236 identifies the blood vessels to be at deeper positions when the difference between the blur amounts of the blood vessel images at different positions is greater. In this way, the position identifying section 230 identifies the position of the objects to be further from the first position 861 and the second position 862 when the blur amount difference is smaller. The image correcting section 220 performs a correction for the blood vessel images of the blood vessel 820 calculated to be deeper by the position identifying section 230 that has a greater effect than the correction performed for the blood vessel image of the blood vessel 810 calculated to be shallower by the position identifying section 230.

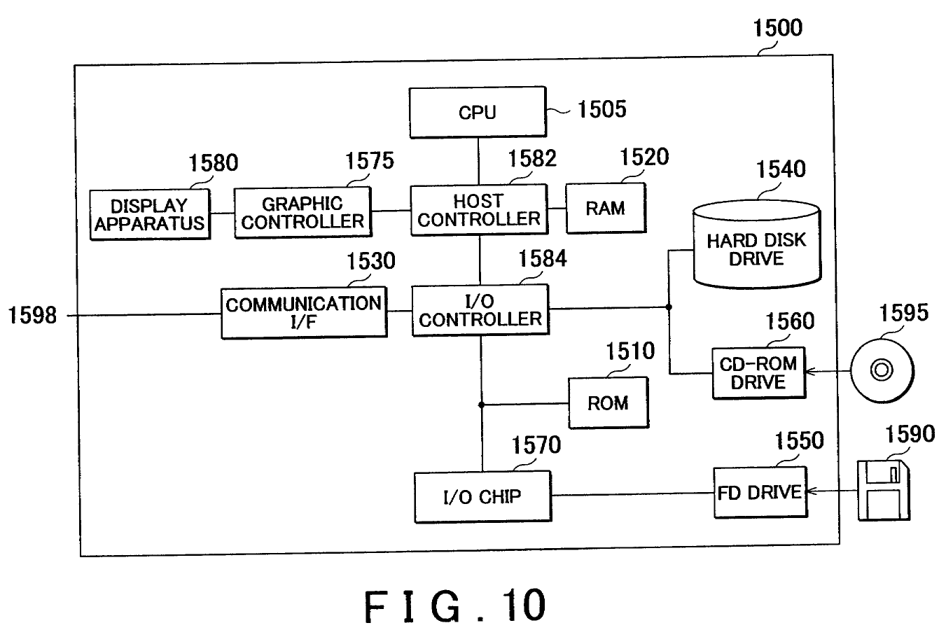

FIG. 10 shows an exemplary hardware configuration of the position identifying system 10 according to the present embodiment. The position identifying system 10 according to the present embodiment is provided with a CPU peripheral section that includes a CPU 1505, a RAM 1520, a graphic controller 1575, and a display apparatus 1580 connected to each other by a host controller 1582; an input/output section that includes a communication interface 1530, a hard disk drive 1540, and a CD-ROM drive 1560, all of which are connected to the host controller 1582 by an input/output controller 1584; and a legacy input/output section that includes a ROM 1510, a flexible disk drive 1550, and an input/output chip 1570, all of which are connected to the input/output controller 1584.

The host controller 1582 is connected to the RAM 1520 and is also connected to the CPU 1505 and graphic controller 1575 accessing the RAM 1520 at a high transfer rate. The CPU 1505 operates to control each section based on programs stored in the ROM 1510 and the RAM 1520. The graphic controller 1575 acquires frame image data generated by the CPU 1505 or the like on a frame buffer disposed inside the RAM 1520 and displays the frame image data in the display apparatus 1580. In addition, the graphic controller 1575 may internally include the frame buffer storing the frame image data generated by the CPU 1505 or the like.

The input/output controller 1584 connects the hard disk drive 1540, the communication interface 1530 serving as a relatively high speed input/output apparatus, and the CD-ROM drive 1560 to the host controller 1582. The communication interface 1530 communicates with other apparatuses via the network. The hard disk drive 1540 stores the programs used by the CPU 1505 in the position identifying system 10. The CD-ROM drive 1560 reads the programs and data from a CD-ROM 1595 and provides the read information to the hard disk drive 1540 via the RAM 1520.

Furthermore, the input/output controller 1584 is connected to the ROM 1510, and is also connected to the flexible disk drive 1550 and the input/output chip 1570 serving as a relatively high speed input/output apparatus. The ROM 1510 stores a boot program performed when the position identifying system 10 starts up, a program relying on the hardware of the position identifying system 10, and the like. The flexible disk drive 1550 reads programs or data from a flexible disk 1590 and supplies the read information to the hard disk drive 1540 and via the RAM 1520. The input/output chip 1570 connects the flexible disk drive 1550 to each of the input/output apparatuses via, for example, a parallel port, a serial port, a keyboard port, a mouse port, or the like.

The programs provided to the hard disk 1540 via the RAM 1520 are stored on a recording medium such as the flexible disk 1590, the CD-ROM 1595, or an IC card and are provided by the user. The programs are read from the recording medium, installed on the hard disk drive 1540 in the position identifying system 10 via the RAM 1520, and are performed by the CPU 1505. The programs installed in and executed by the position identifying system 10 affect the CPU 1505 to cause the position identifying system 10 to function as the components provided to the position identifying system 10 described in relation to FIGS. 1 to 9, such as the image capturing section 110, the vibrating section 133, the image processing section 140, the output section 180, the light irradiating section 150, the control section 105, and the image processing section 140.

While the embodiments of the present invention have been described, the technical scope of the invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the invention.

What is claimed is:

1. A position identifying system that identifies a position of an object existing inside a body, comprising:
a vibrating section that vibrates each of a plurality of different positions inside the body at a different timing;
an image capturing section that captures a frame image of the object at each of the different timings; and
a position identifying section that identifies the position of the object based on a blur amount of an image of the object in each frame image captured by the image capturing section.

2. The position identifying system according to claim 1, wherein
the position identifying section identifies the position of the object as being near a position of the body vibrated by the vibrating section at a timing of the capture of a frame image containing an image of the object having a blur amount greater than a preset value.

3. The position identifying system according to claim 2, wherein
the vibrating section vibrates each of the plurality of different positions in the body at a different timing by generating a plurality of waves, each wave converging at one of the plurality of different positions at a different timing.

4. The position identifying system according to claim 3, wherein
the vibrating section includes a vibration generating section that generates a plurality of vibration waves, each vibration wave converging at one of the plurality of positions from a different direction.

5. The position identifying system according to claim 4, wherein
the vibrating section applies, to the plurality of different positions, a vibration having a vibration component in a direction perpendicular to a direction of the frame image capturing by the image capturing section.

6. The position identifying system according to claim 5, wherein
the image capturing section captures the frame image of the object using light emitted by a luminescent substance inside the object.

7. The position identifying system according to claim 5, wherein
the image capturing section captures the frame image of the object using light reflected from the object.

8. The position identifying system according to claim 5, wherein
the image capturing section captures the frame image of the object using light that passed through the object.

9. The position identifying system according to claim 6, wherein
the position identifying section identifies a depth of the object from a surface of the body, and
the position identifying system further comprises an image correcting section that corrects spread of the image of the object in the frame image obtained by capturing the object, based on the depth identified by the position identifying section.

10. The position identifying system according to claim 9, further comprising a correction table that stores, in association with the depth of the object, a correction value for correcting the spread of the image of the object, wherein
the image correcting section corrects the spread of the image of the object in the frame image obtained by capturing the object, based on the correction value stored in the correction table and the depth of the object.

11. The position identifying system according to claim 10, wherein
the position identifying section identifies the depth of each of a plurality of objects from the surface of the body,
the image correcting section corrects the spread of each of a plurality of images of objects in the frame image, based on the depth of each of the plurality of objects, and
the position identifying system further comprises a display control section that controls display of the frame image corrected by the image correcting section according to the depth of each of the plurality of objects.

12. The position identifying system according to claim 11, wherein
the display control section changes brightness or color of each of the plurality of objects in the frame image corrected by the image correcting section, according to the depth of each object.

13. A position identifying method for identifying a position of an object existing inside a body, comprising:
vibrating each of a plurality of different positions inside the body at a different timing;
capturing a frame image of the object at each of the different timings; and
identifying the position of the object based on a blur amount of an image of the object in each frame image captured during the image capturing.

14. A computer readable medium storing thereon a program causing a position identifying system that identifies a position of an object existing inside a body to function as:
a vibrating section that vibrates each of a plurality of different positions inside the body at a different timing;
an image capturing section that captures a frame image of the object at each of the different timings; and
a position identifying section that identifies the position of the object based on a blur amount of an image of the object in each frame image captured by the image capturing section.

15. A position identifying system that identifies a position of an object existing inside a body, comprising:
a vibrating section that vibrates the body;
an image capturing section that captures a frame image of the object after the body is vibrated; and
a position identifying section that identifies the position of the object inside the body based on a blur amount of an image of the object in the frame image captured by the image capturing section.

16. The position identifying system according to claim 15, wherein the position identifying section further includes:
a transmission time calculating section that calculates a transmission time indicating a period from when the body is vibrated to when the vibration reaches the object, based on the blur amount of the image of object; and
a distance calculating section that calculates a distance from a position at which the body is vibrated to a position of the object, based on the transmission time calculated by the transmission time calculating section.

17. The position identifying system according to claim 16, wherein
the distance calculating section calculates a longer distance when the transmission time calculated by the transmission time calculating section is longer.

18. The position identifying system according to claim 17, wherein
the position identifying section further includes a blur amount calculating section that calculates the blur amount of the image of the object, and
the transmission time calculating section calculates the transmission time to be the period from when the body is vibrated to when the blur amount caused by the vibration becomes greater than a preset value.

19. The position identifying system according to claim 18, wherein
the image capturing section captures the frame image of the object using light emitted by a luminescent substance inside the object.

20. The position identifying system according to claim 18, wherein
the image capturing section captures the frame image of the object using light reflected from the object.

21. The position identifying system according to claim 18, wherein
the image capturing section captures the frame image of the object using light that passed through the object.

22. The position identifying system according to claim 18, wherein
the vibrating section vibrates the surface of the body,
the transmission time calculating section calculates the transmission time to be the period from when the surface is vibrated by the vibrating section to when the blur amount caused by the vibration becomes greater than a preset value, and
the distance calculating section calculates the depth of the object from the surface based on the transmission time calculated by the transmission time calculating section.

23. The position identifying system according to claim 22, further comprising an image correcting section that corrects spread of the image of the object in the frame image obtained by capturing the object, based on the depth of the object.

24. The position identifying system according to claim 23, further comprising a correction table that stores, in association with the depth of the object, a correction value for correcting the spread of the image of the object in the frame image, wherein
the image correcting section corrects the spread of the image of the object in the frame image obtained by capturing the object, based on the correction value stored in the correction table and the depth of the object.

25. The position identifying system according to claim 23, wherein
the transmission time calculating section calculates the transmission time for each of a plurality of objects,
the distance calculating section calculates the depth of each of the plurality of objects from the surface, based on the transmission times calculated by the transmission time calculating section,
the image correcting section corrects the spread of the image of each of the plurality of objects in the frame image, based on the depth of each of the plurality of objects, and
the position identifying system further comprises a display control section that controls display of the frame image corrected by the image correcting section, according to the depth of each object.

26. The position identifying system according to claim 25, wherein
the display control section changes brightness or color of each of the plurality of objects in the frame image corrected by the image correcting section, according to the depth of each object.

27. A position identifying method for identifying a position of an object existing inside a body, comprising:
vibrating the body;
capturing a frame image of the object after the body is vibrated; and
identifying the position of the object inside the body based on a blur amount of an image of the object in the frame image captured by the image capturing section.

28. A computer readable medium storing thereon a program causing a position identifying system that identifies a position of an object existing inside a body to function as:
a vibrating section that vibrates the body;
an image capturing section that captures a frame image of the object after the body is vibrated; and
a position identifying section that identifies the position of the object inside the body based on a blur amount of an image of the object in the frame image captured by the image capturing section.

29. A position identifying system that identifies a position of an object existing inside a body, comprising:
a vibrating section that vibrates the body;
an image capturing section that captures a frame image of the object when the body is vibrated and also when the body is not vibrated; and
a position identifying section that identifies the position of the object inside the body based on a blur amount of an image of the object in the frame image captured by the image capturing section.

30. The position identifying system according to claim 29, wherein
the position identifying section identifies the position of the object inside the body based on a difference between (i) the blur amount of the image of the object when the body is vibrated and (ii) the blur amount of the image of the object when the body is not vibrated.

31. The position identifying system according to claim 30, wherein
the position identifying section identifies the position of the object to be further from the position of the body vibrated by the vibrating section, when the difference between the blur amounts is small.

32. The position identifying system according to claim 31, wherein
the vibrating section vibrates a surface of the body, and
the position identifying section identifies the depth of the object from the surface.

33. The position identifying system according to claim 32, wherein
the vibrating section applies, to the surface, a vibration having a vibration component in a direction perpendicular to a direction of the image capturing by the image capturing section.

34. The position identifying system according to claim 33, wherein
the image capturing section captures the frame image of the object using light emitted by a luminescent substance inside the object.

35. The position identifying system according to claim 33, wherein
the image capturing section captures the frame image of the object using light reflected from the object.

36. The position identifying system according to claim 33, wherein
the image capturing section captures the frame image of the object using light that passed through the object.

37. The position identifying system according to claim 34, further comprising an image correcting section that corrects spread of the image of the object in the frame image obtained by capturing the object, based on the depth identified by the position identifying section.

38. The position identifying system according to claim 37, further comprising a correction table that stores, in association with the depth of the object, a correction value for correcting the spread of the image of the object, wherein
the image correcting section corrects the spread of the image of the object in the frame image obtained by capturing the object, based on the correction value stored in the correction table and the depth of the object identified by the position identifying section.

39. The position identifying system according to claim 38, wherein
the position identifying section identifies the depth of each of a plurality of objects from the surface of the body,
the image correcting section corrects the spread of each of a plurality of images of objects in the frame image, based on the depth of each of the plurality of objects, and
the position identifying system further comprises a display control section that controls display of the frame image corrected by the image correcting section according to the depth of each of the plurality of objects.

40. The position identifying system according to claim 39, wherein
the display control section changes brightness or color of each of the plurality of objects in the frame image corrected by the image correcting section, according to the depth of each object.

41. The position identifying system according to claim 29, wherein
the vibrating section vibrates a first position on a surface of the body and a second position on the surface of the body;
the image capturing section captures the frame image of the object when the first position is vibrated and the second position is not, and also captures the frame image of the object when the second position is vibrated and the first position is not,
the position identifying section identifies the position of the object inside the body based on a difference between (i) a blur amount of the image of the object captured when the first position is vibrated and the second position is not and (ii) a blur amount of the image of the object captured when second position is vibrated and the first position is not.

42. The position identifying system according to claim 41, wherein
the position identifying section identifies the position of the object to be further from the first position and the second position when the difference between the blur amounts is smaller.

43. A method for identifying a position of an object existing inside a body, comprising:
vibrating the body;
capturing a frame image of the object when the body is vibrated and also when the body is not vibrated; and
identifying the position of the object inside the body based on a blur amount of the image of the object in each frame image captured during the image capturing.

44. A computer readable medium storing thereon a program causing a position identifying system that identifies a position of an object existing inside a body to function as:
a vibrating section that vibrates the body;
an image capturing section that captures a frame image of the object when the body is vibrated and also when the body is not vibrated; and
a position identifying section that identifies the position of the object inside the body based on a blur amount of the image of the object in each frame image captured by the image capturing section.
